(19) United States
(12) Patent Application Publication
(10) Pub. No.: US 2025/0032439 A1
(43) Pub. Date: Jan. 30, 2025

(54) NOVEL COMPOSITIONS

(71) Applicant: DSM IP ASSETS B.V., Heerlen (NL)

(72) Inventors: Christine MENDROK-EDINGER, Riehen (CH); Eileen JACKSON, Basel (CH); Angela WILDERMANN, Bad Säckingen (CH); Peter Louis HOUSTON, Boston, MA (US)

(21) Appl. No.: 18/715,405

(22) PCT Filed: Dec. 5, 2022
(86) PCT No.: PCT/EP2022/084470

§ 371 Date: May 31, 2024

Related U.S. Application Data
(60) Provisional application No. 63/285,607, filed on Dec. 3, 2021.

(30) Foreign Application Priority Data
Dec. 21, 2021 (EP) 21216501.3
May 5, 2022 (EP) 22171739.0

(51) Int. Cl. A61K 31/222; A61K 8/67; A61Q 19/00

(52) U.S. Cl. CPC A61K 31/222; A61K 8/671; A61Q 19/00; A61K 2800/10

(57) ABSTRACT

The present invention relates to novel compositions comprising a retinoid mixture of cis and trans isomers of a retinyl ester, particularly retinyl acetate, with a cis/trans ratio of less than 0.03 as well as the use thereof for the treatment, prevention and lessening of the symptoms of diseases and conditions which can be treated with 5-HT2B serotonin receptor antagonists, including but not limited to diseases or conditions selected from migraine headaches, anxiety, ocular hypotension, heart valve disease, irritable bowel syndrome, and MDMA (commonly called “ecstasy”) abuse.

The present invention relates to novel compositions comprising a retinoid mixture of cis and trans isomers of a retinyl ester, particularly retinyl acetate, with a cis/trans ratio of less than 0.03 as well as the use thereof for the treatment, prevention and lessening of the symptoms of diseases and conditions which can be treated with 5-HT2B serotonin receptor antagonists, including but not limited to diseases or conditions selected from migraine headaches, anxiety, ocular hypotension, heart valve disease, irritable bowel syndrome, and MDMA (commonly called “ecstasy”) abuse.

Serotonin 5-HT receptors belong to the superfamily of G protein-coupled seven transmembrane proteins with the exception of the 5-HT3 subtype. They belong to the ligand gated cation superfamily of receptors. G-protein-coupled receptors constitute one of the major signal transduction systems in eukaryotic cells. Serotonin 5-HT2 receptors are widely distributed in the central nervous system and peripheral tissues. The 5-HT2 receptor class couples preferentially to Gq/11 to increase hydrolysis of inositol phosphates and elevate cytosolic [Ca²⁺], which triggers responses in in the body. Serotonin 5-HT2B receptor antagonism is known to be useful for the treatment of migraine headache.

From the seven described 5-HT receptors, 5-HT2B is the only one that signals via inositol trisphosphate and diacylglycerol, both second messengers that activate protein kinase C. In a rat model it has been suggested that protein kinase C gamma activation can sensitize for chronic migraine. Protein kinase C gamma is the protein kinase C isoform that is highly expressed in neuronal tissue.

Serotonin receptor activity also plays a crucial role in anxiety and other illnesses including irritable bowel syndrome, migraine headaches, ocular hypotension, heart valve disease and certain illegal substance abuse (MDMA—commonly called “ecstasy”).

Migraine is a primary headache disorder characterized by recurring headaches which are moderate to severe. They typically affect one side of the head, pulsating in nature, and last from a few hours to three days. Associated symptoms include nausea, vomiting, and sensitivity to light, sound, or smell. The pain is generally made worse by physical activity. Up to one-third of people also experience an aura, i.e., a visual disturbance shortly before the headache appears. The underlying mechanism is not fully known or understood; it is believed to involve the blood vessels and nerves to the brain. Conventional 5 therapy for migraine includes triptan-class pharmaceuticals, such as sumatriptan, although these are not recommended for people with cardiovascular disease. Other therapies address specific symptoms such as pain or nausea. Serotonin 5-HT2B receptor antagonism is also useful for the treatment of migraine headache.

Irritable Bowel Syndrome (IBS) does not appear to have a single cause and there is a wide variety of reasons why patients develop the condition. The single most common trigger to the start of symptom is following a bout of food poisoning or gastroenteritis. Although the cause is not clear, there are many factors that can provoke symptoms. Some people find that stress makes their symptoms worse.

In some people, eating irregularly or eating an abnormal diet may be responsible. Some drugs, particularly when taken long term for chronic conditions, can cause IBS type symptoms such as diarrhea. Overall, there seems to be some interaction between the nervous system in the gut and brain, emotional state and the immune system of the gut.

Approximately one third of IBS patients suffer from bouts of constipation, one third suffer from bouts of diarrhea and most other patients do not fall into a single pattern. Serotonin receptor 5-HT2B antagonists are often used to ameliorate the symptoms.

Ocular hypotension (also referred to as hypotony of the eye) is defined as an intraocular pressure of 5 mm Hg or less. Low intraocular pressure can lead to accelerated cataract formation, maculopathy and discomfort. The 5-HT2B receptor is an important part of the pathway which leads to the development of these conditions.

Problems with the integrity of the function of heart valves has also been linked to the 5-HT2B receptor, and thus can be treated with an antagonist.

MDMA belongs to the phenethylamine family, which further includes amphetamine and other chemically related compounds which act as stimulants, hallucinogens and/or entactogens. MDMA is commonly referred to as “molly” or ecstasy. It works by releasing serotonin (and, to a lesser extent, other neurotransmitters) from their neuroreceptor, and/or blocking their reuptake, thus resulting in increased serotonin levels in the synaptic cleft. The excess release of serotonin by MDMA is the likely cause of the mood-elevating effect that the user experiences. However, the release of a large amount of serotonin by MDMA also causes the brain to become significantly depleted of this neurotransmitter, and this contributes to the negative psychological aftereffects that are experienced for several days after taking MDMA. Administration of a mixture and/or composition comprising said mixture according to the present invention prevents the initial release of serotonin, thus dulling or negating the mood-elevating experience, and lessening the serotonin-depleting phase, thus lessening the appeal of the drug. Unfortunately, most of the medications using known 5-HT2B antagonists are associated with negative side effects and are not recommended for long-term therapy.

Thus, it would be desirable to have an alternative therapy to prevent, treat and mitigate the symptoms of migraine, ocular hypotension, heart valve disease, irritable bowel syndrome, and certain illegal substance abuse (MDMA--commonly called “ecstasy”), i.e. symptoms that are related to the 5-HT2B pathway.

Surprisingly, it has been found that certain mixtures of cis and trans retinyl esters, i.e. retinoid mixtures comprising cis and trans-isomers of retinyl esters, particularly mixtures comprising certain ratios of cis/trans retinyl acetate, with a cis/trans ratio of less than about 0.03 are particularly suitable to bind to and antagonize the 5-HT2B serotonin receptor, and thus can be used to treat, prevent, or lessen the symptoms of a disease associated with 5-HT2B overactivity. Such diseases include migraine, anxiety, irritable bowel syndrome, ocular hypotension, heart valve disease, and MDMA (commonly called “ecstasy”) abuse.

Thus, the present invention relates to a retinoid mixture comprising cis and trans retinyl esters, particularly cis and trans-retinyl acetate, with a cis/trans ratio of less than 0.03, particularly wherein the cis/trans ratio is in the range of about 0.0025.

Particularly, the present invention relates to a pharmaceutical, food, feed or cosmetic composition comprising said retinoid mixture comprising cis and trans isomers of a retinyl ester, particularly cis/trans isomers of retinyl acetate, with a cis/trans ratio of less than 0.03, including pharmaceutical, food, feed or cosmetic compositions or retinoid mixtures as defined herein comprising a cis/trans ratio of retinyl acetate different from commercially available all-trans retinyl acetate, such as e.g. Dry Vitamin A-Acetate 500 (BASF) or Vitamin A Acetate crystalline (DSM).

“Retinyl esters” as used herein refer to compounds of formula (1) (without any indication of the stereochemistry):

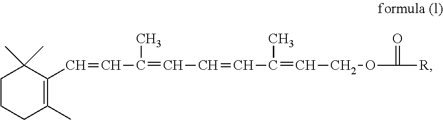

wherein
R is selected from alkyl groups with 2 to 18 carbon atoms including the ester carbonyl, preferably from methyl, ethyl, propyl, butyl, stearyl and palmityl. In all embodiments of the present invention, most preferably R is methyl, i.e. the compound of formula (1) is retinyl acetate.

The cis/trans ratio as given herein refers to the wt.-% ratio of the sum of all cis-isomers to the respective all trans isomer of the respective retinoid, such as e.g. retinyl acetate, retinol, retinal or the like, as determined by known methods such as e.g. HPLC, assuming the same response factor for all isomers.

As used herein, a “retinoid mixture” comprises retinyl ester isomers, particularly retinyl acetate isomers, such as cis- and trans-isomers, in a ratio as defined herein. The retinoid mixture might comprise cis and/or trans isomers of retinol or retinal.

As used herein, the cis/trans ratio, particularly the ratio of cis-isomers to trans-isomers in a retinoid mixture comprising retinyl acetate, retinol or retinal, more particularly the ratio of cis-retinyl acetate to trans-retinyl acetate or the ratio of cis-retinol to trans-retinol or the ratio of cis-retinal to trans-retinal, means comparing the wt % of a cis-isomer to the wt % of the corresponding trans-isomer, all based on total retinoids in said mixture. Thus, a mixture comprising a cis/trans ratio of 0.03 with regards to retinyl acetate is a mixture with e.g. 1 wt % cis-retinyl acetate based on total retinoids within said mixture and 33 wt % trans-retinyl acetate based on total retinoids within said mixture. A cis/trans ratio of 0.03 with regards to retinyl acetate would also apply to a mixture with 2.95 wt % cis-retinyl acetate and 94.15% trans-retinyl acetate, or with 2.99 wt % cis-retinyl acetate and 99.6% trans-retinyl acetate, or with 2.98 wt % cis-retinyl acetate and 99.2 wt % trans-retinyl acetate, or with 2.97 wt % cis-retinyl acetate and 99.7 wt % trans-retinyl.

As used herein, the term “trans-retinyl acetate”, “trans-retinol”, “trans-retinal” or the like are known to the skilled person and in line with the IUPAC-IUB nomenclature and mean that all double bonds in such retinyl acetate, retinol, retinal compound, including the compound according to formula (1), are in trans configuration. The term “trans-retinyl ester”, particularly “trans-retinyl acetate”, and “all-trans-retinyl ester”, particularly “all-trans-retinyl acetate”, are used interchangebly herein. Thus, if one of several double bonds is not in the trans configuration but in the cis configuration, such isoforms are named herein as e.g. cis-retinyl acetate or cis-retinol or cis-retinal.

Retinoid isomers in cis-configuration that are covered by the present invention include but are not limited to 9-cis-retinol, 11-cis retinol, 13-cis retinol, 9,13-di-cis-retinol, 11,13-di-cis retinol, 13-cis-3,4-didehydro retinol, 9-cis-3,4-didehydro retinol, 9,13-di-cis-3,4-didehydro retinol, 13-cis retinal, 11-cis-retinal, 11,13-di-cis retinal, 9,13-di-cis-retinal, 9-cis retinal, 13-cis-3,4-didehydro retinal, 11,13-di-cis-3,4-didehydro retinal, as well as the respecitve cis-forms of retinyl acetate (for review, see e.g. Table 1 in Gundersen and Blomhoff, J. Chromatogr. A 935,13-43, 2001).

In all embodiments of the present invention the cis/trans ratio of retinyl ester isomers as defined above, i.e. wt % of cis- or trans-retinyl ester, particularly retinyl acetate, based on total retinoids in the mixture, is less than 0.03, preferably a cis/trans ratio of 0.0299, 0.028, 0.025, 0.02, 0.015, 0.01, 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0299 to 0.0001, 0.01 to 0.001, 0.02 to 0.0005, 0.0099 to 0.0001, 0.008 to 0.001, 0.007 to 0.002, 0.009 to 0.0001, 0.008 to 0.0001, 0.005 to 0.001, 0.005 to 0.0001, 0.005 to 0.0005, 0.006 to 0.001, 0.02 to 0.0001, 0.01 to 0.0001, 0.006 to 0.0001, 0.0055 to 0.0001, 0.007 to 0.0001, 0.0045 to 0.0001, 0.005 to 0.002, 0.004 to 0.0001, 0.0035 to 0.0001, 0.003 to 0.0001, 0.005 to 0.0025, 0.025 to 0.0001, 0.006 to 0.0005, 0.006 to 0.0008, 0.006 to 0.0015, 0.006 to 0.002, 0.006 to 0.003, 0.006 to 0.0004, 0.006 to 0.0025, 0.004 to 0.0005, most preferably a cis/trans ratio of about 0.0025.

According to further inventive embodiments, the cis/trans ratio of retinyl ester isomers, particularly retinyl acetate isomers as defined above, i.e. wt % of cis- or trans-retinyl ester, particularly retinyl acetate, based on total retinoids in the mixture, is 1:more than 33.33, preferably a cis/trans ratio of 1:35.72, 1:40, 1:45, 1:50, 1:55, 1:60, 1:65, 1:66.67, 1:70, 1:75, 1:80, 1:90, 1:95, 1:100, 1:101.01, 1:105.26, 1:110, 1:111.11, 1:115, 1:117.65, 1:120, 1:125, 1:130, 1:133.33, 1:135, 1:140, 1:142.86, 1:145, 1:150, 1:155, 1:160, 1:165, 1:166.67, 1:170, 1:175, 1:180, 1:181.82, 1:185, 1:190, 1:195, 1:200, 1:204.08, 1:205, 1:210, 1:215, 1:220, 1:222.22, 1:225, 1:230, 1:232.56, 1:235, 1:240, 1:245, 1:250, 1:255, 1:260, 1:265, 1:270, 1:275, 1:280, 1:285, 1:285.71, 1:290, 1:295, 1:300, 1:305, 1:310, 1:315, 1:320, 1:325, 1:330, 1:333.33, 1:335, 1:340, 1:345, 1:350, 1:355, 1:357.14, 1:360, 1:365, 1:370, 1:375, 1:380, 1:385, 1:390, 1:395 or 1:400, 1:405, 1:410, 1:420, 1:430, 1:440, 1:450 or 1:500, 1:1000 or less, more preferably ranges of1:33 to 1:1000, 1:100 to 1:500, 1:100 to 1:1000, 1:125 to 1:1000, 1:200 to 1:1000, 1:200 to 1:500, 1:200 to 1:400, most preferably a cis/trans ratio of about 1:400.

The retinoid mixture comprising cis and trans isomers of retinyl esters, particularly mixtures of cis and trans isomers of retinyl acetate, and or pharmaceutical, food, feed or cosmetic compositions comprising said mixtures according to the present invention can either be prepared by admixing the respective all-trans isomer with one or more cis isomers obtained by chemical or biological processes. Methods to prepare such all trans and/or cis isomers are well known to a person skilled in the art. Alternatively, the mixture can be prepared in said isomer ratio by adjusting the processes accordingly.

Advantageously, in all embodiments of the present invention, the retinoid mixtures comprising retinyl esters, particularly retinyl acetate, are biologically produced through a fermentation process. In this process trans retinyl esters, in particular trans retinyl acetate, are made through the action of specific enzymes that results in stable all trans retinyl esters, preferably all trans retinyl acetate, being produced. The trans retinyl ester, preferably trans retinyl acetate, can be treated with heat to form cis retinyl esters, preferably cis retinyl acetate, to achieve the proper levels mentioned previously in all the embodiments of the present invention (see e.g. McBee et al., JBC, Vol. 276, No. 51, pp. 48483-48493, 2001).

Advantageously, in all embodiments of the present invention, the retinoid mixture comprising cis and trans retinyl esters, particularly retinyl acetate, with a cis/trans ratio of less than 0.03, particularly wherein the cis/trans ratio is in the range of about 0.0025, are biologically produced through a fermentation process, preferably leading to a retinoid mixture with yellowish color.

As known in the art, color can be precisely described in several different co-ordinate systems, such as XYZ, RGB, CYMK, or L*a*b* (CIELAB according to EN ISO/CIE 11664-4: 2019). A preferred method is the definition via the L*a*b* system. The skilled person knows which instrument to use depending on the different color measurement systems and how to measure the color of the mixtures as described herein.

A “yellowish” color as used herein means a color as defined by the L*a*b* color system, particularly wherein L*a*b* being (3<L*<100, -25<a*<30,10<b*<150), such as e.g. L* being in the range of 50 to 100, a* being in the range of −25 to 10 and b* being in the range of 40 to 150, preferably wherein L* being in the range of 80 to 100, a* being in the range of −22 to 1, b* being in the range of 40 to 85 or 100 to 140, more preferably L* being in the range of 80 to 95, a* being in the range of −17 to −1, b* being in the range of 105 to 135, particularly L* being about 80, 81, 82, 83, 84, 85, 86, 87, 88, 89, 90, 91, 92, 93, 94, 95, 96, 97, 98, 99, 100 and a* being about −24.9, −24, −23, −22, −21, −20, −19, −18, −17, −18, −17, −16, −15, −14, −13, −12, −11, −10, −9, −8, −7, −6, −5, −4, −3, −2, −1, 0 and b* being about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135.

In a particular embodiment, the retinoid mixture comprising cis and trans isomers of retinyl esters, particularly retinyl acetate as defined herein, are produced in a fermentation process using suitable retinyl ester/acetate producing host cells, such as e.g. bacterial or fungal cells, (see e.g. Sun et al, ACS Synth. Biol. 2019 Sep. 20; 8(9):2131-2140; Jang et al., Microbial Cell Factories 2011, 10:59), wherein the cells are expressing the respective enzymes, such as e.g. enzymes involved in biosynthesis of retinyl esters, particularly retinyl acetate, from conversion of retinol. Particularly, the fermentation is fed ethanol, corn sugar or corn oil all derived from agricultural production. The fermentation products comprising retinyl esters, particularly retinyl acetate, might be extracted in an aliphatic phase and subsequently purified to crystalline forms by methods as used in the art.

In some embodiments, mixtures of retinoids as defined herein, particularly mixtures comprising retinyl acetate with specific cis/trans ratio according to the present invention are obtained in a fermentation process with retinoid-producing host cells, wherein beta-carotene is enzymatically converted into retinoids comprising retinal, retinol or retinyl acetate with a cis/trans ratio as defined herein, wherein the inventive cis-/trans ratio can be obtained using one or more enzymes having oxygenase and isomerase functionality (see e.g. Oberhauser et al., PNAS, vol 105, no. 48, 19003, 2008). Particularly, the retinoid-producing host organism is capable of enzymatically converting beta-carotene into retinyl acetate via retinal and retinol.

The term “biologically produced” as used herein means that the retinyl ester, particularly retinyl acetate, is produced by the help of biotechnological process such as a fermentation process including cultivation of a suitable (carotenoid and/or retinoid producing) host cell expressing the respective enzymes involved in conversion of a suitable carbon source into retinyl esters, particularly retinyl acetate, as defined herein, wherein the host cell might be selected from bacteria, fungi, particularly yeast, plant or algae, particularly from fungal cells, such as e.g. Yarrowia or Saccharomyces, or bacterial cells, such as e.g. E. coli. It might furthermore be selected from other suitable microorganism known to produce retinoids. “Bio-produced”, “biologically-derived” and “biologically produced” or “bio-based” are used synonymously herein. Accordingly, said biologically produced retinoids comprising retinyl ester, particularly retinyl acetate, are composed of carbon from atmospheric carbon dioxide (also referred to as carbon of atmospheric origin) converted to sugars and starches by green plants and thus preferably sugars and starches composed of non-fossil-fuel carbon. It also includes the use of isolated and/or immobilized enzymes in a process for generation of the retinyl ester, particularly retinyl acetate, mixture as defined herein, such as specific enzymes capable of selectively catalyzing the formation of the specific trans/cis ratio of retinyl esters, particularly retinyl acetate, in a mixture as defined herein.

In one aspect, the retinoid mixture as defined herein is produced via fermentation using a fungal host cell, such as e.g. an oleaginous yeast, particularly Yarrowia, said host cell being cultivated in the presence of a suitable carbon source and in the presence of a plant-based second phase, e.g. vegetable oil, such as e.g. corn oil, as second phase solvent, wherein the retinoids are accumulated in the second phase.

Suitable plant-based second phase solvents might be selected from any vegetable oil, including but not limited to oleic, palmitic, steric or linoleic acid and glycerol, such as e.g. corn, olive, cottonseed, rapeseed, sesame, canola, safflower, sunflower, soybean, grapeseed, or peanut oil, preferably corn oil.

Carbon sources to be used for the present invention might be selected from linear alkanes, free fatty acids, ethanol, glucose and/or mixtures thereof.

As used herein, a “bio-based” compound has a C-14/C-12 isotope ratio in the range of from 1:0 to greater than 0:1, as in contrast to a fossil-based compound with a C-14/C-12 isotope ratio of 0:1. Bio-based content of a compound can be measured by known radiocarbon and isotope ratio mass spectrometry analysis or accelerator mass spectrometry, such as e.g. the ASTM test method D6866 such as D6866-05 or D6866-20, DIN SPEC 91236 (CEN/TS 16137), ISO 16620, or DIN EN 16785/1, wherein the C-14/C-12 isotope ratio in a sample is measured and compared to a standard 100% biobased material to give percent biobased content of the sample.

“Carbon of atmospheric origin” as used herein refers to carbon atoms from carbon dioxide molecules that have recently, in the last few decades, been free in the earth's atmosphere. Such carbons in mass are identifiable by the presence of particular radioisotopes as described herein. “Green carbon”, “atmospheric carbon”, “environmentally friendly carbon”, “life-cycle carbon”, “non-fossil fuel-based carbon”, “non-petroleum based carbon”, “carbon of atmospheric origin”, and “biobased carbon” are used interchangeably herein.

In all embodiments of the present invention, advantageously the retinoid mixture comprising retinyl ester, particularly retinyl acetate, is produced by merely organic, renewable, bio-based feedstock, particularly fermentatively produced, as such retinyl ester, particularly retinyl acetate, has an anthropogenic CO₂ emission profile of zero upon biodegradation because all of the CO₂ molecules released during degradation from such “fermentatively-derived” or “fermentatively-produced” retinyl esters, particularly retinyl acetate, have an atmospheric origin. Thus, the net release of CO₂ to the atmosphere is zero.

Thus, the present invention is particularly directed to a retinoid mixture comprising retinyl acetate comprising cis- and trans-isomers as defined herein, with a cis-/trans ratio (defined as wt % based on total retinoids in the mixture) of less than 0.03, wherein said mixture has at least about 20%, such as 25, 30, 35, 40, 45, 50, 55, 60, 65, 70, 75, 80, 85, 90, 95, 98 or 100% biobased carbon according to the definition given in the present application.

Particularly, the present invention is directed to a biodegradable composition comprising a mixture of retinyl acetate with a cis-/trans-ratio of less than 0.03 as defined herein, wherein the retinyl acetate mixture in said composition has an anthropogenic CO₂ emission profile of zero upon biodegradation.

In one preferred embodiment the retinoid mixture according to the present invention consists essentially of cis- and trans-retinyl esters, particularly cis and trans retinyl acetate, in the ranges and with all the definitions and preferences as given herein.

The term “consists essentially of” as used according to the present invention means that the total amount of ingredients within the mixture as defined herein ideally sum up to 100 wt.-%. It is however not excluded that small amounts of impurities or additives may be present in the mixture, with the proviso that the total amount of such impurities is preferably less than about 5 wt.-%, more preferably less than about 3, 2.1 wt.-% and which are e.g. introduced via the respective raw materials and/or processes used.

As used herein, the term “impurities” and “additives” are used interchangeably herein and refer to co-ingredients within the inventive mixture and that are present in the inventive mixture in an amount of less than 5 wt.-% based on all ingredients present in the mixture.

If the retinoid mixture according to the present invention consists essentially of retinyl acetate with a cis/trans ratio as defined herein, then the purity of said mixture, i.e. the (total) amount of cis and trans retinyl acetate is preferably at least about 95%, more preferably at least about 96%, most preferably at least about 98% or more, as determined by known methods such as e.g. HPLC, particularly reversed phase C4 HPLC.

As used herein, the term “purity” means the sum of cis- and trans isomers in relation to the total weight, e.g. the sum of cis-retinyl acetate and trans-retinyl acetate according to the definition given herein gives a percentage of purity based on total retinoids set to 100% purity. Thus, if a mixture consists essentially of 98.03 wt % trans-retinol and 0.25 wt % cis-retinol, said mixture has a purity with regards to retinol of 98.28%. Preferably, and in accordance with all embodiments of the present invention, the retinoid mixture comprises less than 0.5 wt % cis-retinyl esters, particularly cis-retinyl acetate, based on total retinoids in said mixture.

In some embodiments, the retinoid mixtures comprising cis and trans retinyl acetate with a cis/trans ratio as defined herein further comprise small amounts of additives such as e.g. dihydro-retinoids, including dihydro-retinol and/or dihydro-retinyl acetate, particularly in a range of 0.2 to 0.01 wt % or less, such as e.g. 0.2, 0.18, 0.16, 0.15, 0.14, 0.12, 0.1, 0.05, 0.01 wt % or less, preferably a range of 0.2 to 0.1, 0.17 to 0.06, 0.1 to 0.05, 0.04 to 0.01, more preferably a percentage of 0.1 wt % or less, all based on total retinoids. Also preferably, the percentage of dihydro-retinyl acetate is about 0.05 wt % or less, particularly about 0.01 wt % or less based on total retinoids within said mixture.

In some embodiments, the retinoid mixtures comprising cis and trans retinyl acetate with a cis/trans ratio as defined herein further comprise small amounts of additives such as e.g. retinol, including but not limited to cis-retinols, trans-retinol, dihydro-retinol, such as in amounts of 2 wt % or less, such as about 1.8, 1.7, 1.5, 1.2, 1.0, 0.8, 0.5, 0.3, 0.2, 0.1 wt % or less, preferably percentage of retinol in the range of 2 to 0.1 wt %, 2 to 1 wt %, 1 to 0.1 wt %, 1.5 to 0.1 wt %, 0.5 to 0.2 wt %, 0.4 to 0.2 wt %, 1 to 0.2 wt %, most preferably a range of 2 to 0.9 wt % or 0.4 to 0.1 wt % all based on total retinoids within said mixture.

In some embodiments, the retinoid mixtures comprising cis and trans retinyl acetate with a cis/trans ratio as defined herein further comprise small amounts of additives such as e.g. retinal, including but not limited to 9-cis retinal, trans retinal, such as in amounts of 1 wt % or less, such as about 0.8, 0.7, 0.5, 0.2, 0.1, 0.08, 0.05, 0.03, 0.02, 0.01, 0.005 wt % or less, preferably percentage of retinal in the range of 1 to 0.005 wt %, 0.5 to 0.01 wt %, 0.5 to 0.02 wt %, 0.05 to 0.01 wt %, 0.05 to 0.02 wt %, 0.04 to 0.1 wt %, 0-2 to 0.04 wt %, most preferably a range of 0.2 to 0.019 wt % or 0.04 to 0.01 wt % all based on total retinoids within said mixture.

Another embodiment of this invention is a method of treating or preventing an adverse condition which is responsive to antagonizing the serotonin 5-HT2B receptor comprising administering an effective amount of a retinoid mixture and/or pharmaceutical, food, feed or cosmetic composition comprising said mixture according to the present invention with all the definitions and preferences as given herein to a person in need thereof. Preferably the adverse condition is selected from the group consisting of migraine, anxiety, ocular hypotension, heart valve disease, irritable bowel syndrome, and addiction/abuse of a member of the phenethylamine family of stimulants, particularly methylenedioxymethamphetamine (MDMA).

Another embodiment is the use of a retinoid mixture and/or pharmaceutical, food, feed or cosmetic composition comprising said mixture according to the present invention with all the definitions and preferences as given herein to treat or prevent an adverse condition which is responsive to antagonizing the serotonin 5-HT2B receptor. Another embodiment of this invention is the use of a retinoid mixture and/or composition according to the present invention with all the definitions and preferences as given herein in the manufacture of a nutraceutical, dietary, food, feed, pharmaceutical or cosmetic product including a medicament to treat or prevent and adverse condition which is responsive to antagonizing the serotonin 5-HT2B receptor.

Thus, the present invention is directed to the use of a retinoid mixture and/or pharmaceutical, food, feed or cosmetic composition comprising said mixture with a certain cis/trans ratio of retinyl esters, particularly retinyl acetate, as defined herein, said mixture/composition being used as 5-HT2B receptor antagonist, particularly for the treatment or prevention or lessening of symptoms or adverse conditions in connection with migraine, anxiety, ocular hypotension, heart valve disease, irritable bowel syndrome, and addiction/abuse of a member of the phenethylamine family of stimulants, particularly methylenedioxymethamphetamine (MDMA).

In all embodiments of the present invention the application can be topical or oral, oral being preferred.

It is well understood in all embodiments of the present invention, that the adverse condition may also encompass adverse skin conditions. “Prevention” or “prophylaxis” includes not only total prevention, delaying the onset of, lessening the severity of symptoms of the condition, and/or increasing the length of time between recurrence of the condition.

Another embodiment of this invention is a method of treating, prevention, or lessening the symptoms of a migraine headache comprising administering a 5-HT2B receptor antagonizing-amount of a retinoid mixture and/or pharmaceutical, food, feed or cosmetic composition comprising said mixture according to the present invention with all the definitions and preferences as given herein, preferably comprising the step of administering such mixture and/or composition to a person experiencing a migraine headache or at risk of experiencing a migraine headache.

A further embodiment is the use of a retinoid mixture and/or pharmaceutical, food, feed or cosmetic composition comprising said mixture according to the present invention with all the definitions and preferences as given herein to treat, prevent or lessen the severity of a migraine headache.

Another embodiment is the use of a retinoid mixture and/or pharmaceutical, food, feed or cosmetic composition comprising said mixture according to the present invention with all the definitions and preferences as given herein according to the present invention with all the definitions and preferences as given herein in the manufacture of a nutraceutical, dietary, pharmaceutical, feed, food or cosmetic product including a medicament to treat, prevent, or lessen the symptoms of a migraine, particularly migraine headache.

In another embodiment, the retinoid mixture and/or pharmaceutical, food, feed or cosmetic composition comprising said mixture as defined herein with all the definitions and preferences as given herein is used for the treatment, prevention or lessening of symptoms associated with anxiety.

Another embodiment of this invention is a method of treating, prevention, or lessening the symptoms of anxiety comprising administering a 5-HT2B receptor antagonizing-amount of a retinoid mixture and/or pharmaceutical, food, feed or cosmetic composition comprising said mixture according to the present invention with all the definitions and preferences as given herein.

Another embodiment is the use of a retinoid mixture and/or pharmaceutical, food, feed or cosmetic composition comprising said mixture according to the present invention with all the definitions and preferences as given herein in the manufacture of a nutraceutical, dietary, pharmaceutical, food, feed or cosmetic product including a medicament to treat, prevent, or lessen the symptoms of anxiety.

Another embodiment of this invention is a method of treating, prevention, or lessening the symptoms of Irritable Bowel Syndrome (IBS) comprising administering a 5-HT2B receptor antagonizing-amount of a retinoid mixture and/or pharmaceutical, food, feed or cosmetic composition comprising said mixture according to the present invention with all the definitions and preferences as given herein.

A further embodiment is the use of a retinoid mixture and/or pharmaceutical, food, feed or cosmetic composition comprising said mixture according to the present invention with all the definitions and preferences as given herein to treat, prevent or lessen IBS. Another embodiment is the use of a said retinoid mixture or composition according to the present invention with all the definitions and preferences as given herein in the manufacture of a nutraceutical, dietary, food, feed, pharmaceutical or cosmetic product including a medicament to treat, prevent, or lessen the symptoms of IBS. A particularly preferred retinyl ester is retinyl acetate.

Another embodiment of this invention is a method of treating, prevention, or lessening the symptoms of ocular hypotension, including the formation of cataracts, comprising administering a 5-HT2B receptor antagonizing-amount of a retinoid mixture and/or pharmaceutical, food, feed or cosmetic composition comprising said mixture with a certain cis/trans ratio according to the present invention with all the definitions and preferences as given herein.

A further embodiment is the use of a retinoid mixture and/or pharmaceutical, food, feed or cosmetic composition comprising said mixture according to the present invention with all the definitions and preferences as given herein to treat, prevent or lessen ocular hypotension, including cataracts.

A further embodiment is the use of pharmaceutical, food, feed or cosmetic composition according to the present invention with all the definitions and preferences as given herein in the manufacture of a nutraceutical, dietary, pharmaceutical, feed, food or cosmetic product including a medicament to treat, prevent, or lessen the symptoms of ocular hypotension, including cataracts.

Another embodiment of this invention is a method of treating, prevention, or lessening the symptoms of heart valve disease, comprising administering a 5-HT2B receptor antagonizing-amount of a retinoid mixture and/or pharmaceutical, food, feed or cosmetic composition comprising said mixture according to the present invention with all the definitions and preferences as given herein.

A further embodiment is the use of a product comprising a retinoid mixture and/or pharmaceutical, food, feed or cosmetic composition comprising said mixture as defined herein to treat, prevent or lessen a symptom of heart valve disease.

Another embodiment is the use of a retinoid mixture and/or pharmaceutical, food, feed or cosmetic composition comprising said mixture according to the present invention with all the definitions and preferences as given herein in the manufacture of a nutraceutical, dietary, food, feed, pharmaceutical or cosmetic product including a medicament to treat, prevent, or lessen the symptoms of a heart valve disease.

One embodiment of this invention is a method of preventing, treating or lessening symptoms associated with phenethylamine drug dependency comprising administering a composition comprising a serotonin 5-HT2B—antagonizing amount of retinoid mixture and/or pharmaceutical, food, feed or cosmetic composition comprising said mixture according to the present invention with all the definitions and preferences as given herein to a person in need thereof, or at risk or abusing drugs causing excess release of serotonin, including but not limited to drugs of the phenethylamine family, preferably methylenedioxymethamphetamine (MDMA).

Another embodiment is the use of a pharmaceutical, food, feed or cosmetic composition according to the present invention with all the definitions and preferences as given herein to lessen the appeal of a phenylethylamine drug for a person who abuses the drug. In preferred embodiments, the drug is MDMA.

Another embodiment is the use of a retinoid mixture and/or pharmaceutical, food, feed or cosmetic composition comprising said mixture according to the present invention with all the definitions and preferences as given herein in the manufacture of a nutraceutical, dietary, pharmaceutical or cosmetic product including a medicament to prevent or treat abuse of phenylethylamine drug. In preferred embodiments, the drug is MDMA.

The retinoid mixture and/or pharmaceutical, food, feed or cosmetic composition comprising said mixture according to the present invention with all the definitions and preferences as given herein may be combined with other active ingredients to make a product which has beneficial results. Examples of further active ingredients include Vitamin E, water soluble tomato extract, resveratrol, Vitamin D, 25-hydroxy vitamin D3, hydroxytyrosol, polyunsaturated fatty acids (PUFAs), human milk oligosaccharides, cannabinoids, and mixtures thereof.

The products according to the present invention are generally prepared by admixing a retinoid mixture and/or pharmaceutical, food, feed or cosmetic composition comprising said mixture according to the present invention with suitable carriers and/or excipients and/or diluents conventionally used in nutraceutical, pharmaceutical, dietary or cosmetic products, with a daily recommended dose of a sufficient amount of a retinoid mixture comprising retinyl acetate, with a cis/trans ratio of less than 0.03 as defined herein, particularly up to 2 mg retinly acetate per day for an adult.

The term “cosmetic product” as used in the present application refers to compositions as defined under the heading “Kosmetika” in Römpp Lexikon Chemie, 10th edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4^{th} edition, 1992.

For combinations of the active ingredients, the dosages may be adjusted so that the dosages of the combined ingredients within the nutraceutical, dietary, pharmaceutical, feed, food, or cosmetic products are from at least 0.1 to 10 mg per day, but should not exceed 30 mg per day.

If desired, the daily intake can be divided into two or more dosages, such as twice a day tablets.

For non-human animals, the human dosages above can be adjusted to the animal's body weight.

The nutraceutical, dietary, feed, food, pharmaceutical or cosmetic products comprising a retinoid mixture and/or pharmaceutical, food, feed or cosmetic composition comprising said mixture according to the present invention with all the definitions and preferences as given herein may be in any galenic form that is suitable for administering to the human or animal body, especially in any form that is conventional for oral administration, e.g. in solid form, such as (additives/supplements for) food or food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be encapsulated in hard or soft shell capsules, whereby the capsules feature e.g. a matrix of (fish, swine, poultry, cow) gelatin, plant proteins or ligninsulfonate. Examples for other application forms are forms for topical, transdermal, parenteral or injectable administration. The nutraceutical, dietary, pharmaceutical, food, feed, or cosmetic products may be in the form of controlled (delayed) release formulations. The nutraceutical, dietary, pharmaceutical, food, feed or cosmetic products according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

Examples of food which is suitable for containing the pharmaceutical, food, feed or cosmetic composition according to the present invention with all the definitions and preferences as given herein are cereal bars, dairy products, such as yoghurts, and bakery items, such as cakes and cookies. Examples of fortified food are cereal bars, and bakery items, such as bread, bread rolls, bagels, cakes and cookies. Examples of dietary supplements are tablets, pills, granules, dragées, capsules and effervescent formulations, in the form of non-alcoholic drinks, such as soft drinks, fruit juices, lemonades, near-water drinks, teas and milk-based drinks, in the form of liquid food, such as soups and dairy products (muesli drinks). Also appropriate are beverages. Beverages encompass non-alcoholic and alcoholic drinks as well as liquid preparations to be added to drinking water and liquid food. Non-alcoholic drinks are e.g. soft drinks, sport drinks, fruit juices, vegetable juices (e.g. tomato juice), lemonades, teas and milk-based drinks. Liquid foods are e.g. soups and dairy products (e.g. muesli drinks).

In addition to the retinoid mixture and/or pharmaceutical, food, feed or cosmetic composition comprising said mixture according to the present invention with all the definitions and preferences as given herein, the nutraceutical, dietary, food, feed, pharmaceutical or cosmetic products according to the present invention may further contain conventional additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like used in the respective field. The carrier material can be organic or inorganic inert carrier material suitable for oral/parenteral/injectable/topical administration.

The following embodiments (1) to (14) are particularly within the scope of the present invention:
(1): A mixture of cis and trans isomers of retinyl ester, wherein the cis/trans ratio within said mixture is less than 0.03, preferably a cis/trans ratio of 0.0299, 0.028, 0.025, 0.02, 0.015, 0.01, 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0299 to 0.0001, 0.01 to 0.001, 0.02 to 0.0005, 0.0099 to 0.0001, 0.008 to 0.001, 0.007 to 0.002, 0.009 to 0.0001, 0.008 to 0.0001, 0.005 to 0.001, 0.005 to 0.0001, 0.005 to 0.0005, 0.006 to 0.001, 0.02 to 0.0001, 0.01 to 0.0001, 0.006 to 0.0001, 0.0055 to 0.0001, 0.007 to 0.0001, 0.0045 to 0.0001, 0.005 to 0.002, 0.004 to 0.0001, 0.0035 to 0.0001, 0.003 to 0.0001, 0.005 to 0.0025, 0.025 to 0.0001, 0.006 to 0.0005, 0.006 to 0.0008, 0.006 to 0.0015, 0.006 to 0.002, 0.006 to 0.003, 0.006 to 0.0004, 0.006 to 0.0025, 0.004 to 0.0005, most preferably a cis/trans ratio of about 0.0025.
(2): A composition comprising the mixture of embodiment (1).
(3): Mixture of embodiment (1) or composition of embodiment (2) wherein the retinyl ester is retinyl acetate.
(4): Mixture/composition of embodiments (1), (2), (3), wherein the retinyl ester is biologically-derived.
(5): Mixture/composition of embodiments (1), (2), (3) or (4), wherein the mixture consists of at least about 95% retinyl acetate based on all components in the mixture.
(6): Mixture/composition of embodiments (1), (2), (3), (4) or (5), comprising a percentage of up to about 2% of retinol based on total components in said mixture.
(7): A method of preventing or treating an adverse condition which is responsive to antagonizing the serotonin 5-HT2B receptor comprising administering an effective amount of a mixture/composition of embodiments (1), (2), (3), (4), (5) or (6) to a person in need thereof.
(8): Method of embodiment (7) for prevention, treatment or lessening of symptoms associated with migraine, anxiety, irritable bowel syndrome, ocular hypotension, heart valve disease, and addiction/abuse of a member of the phenethylamine family of stimulants.
(9): Use of a mixture/composition of embodiments (1), (2), (3), (4), (5) or (6) as serotonin 5-HT2B receptor antagonist.
(10): Use according to embodiment (9), wherein said 5-HT2B receptor antagonist is used for the treatment or prevention of migraine, anxiety, irritable bowel syndrome, ocular hypotension, heart valve disease, and addiction/abuse of a member of the phenethylamine family of stimulants.
(11): Use of a mixture or composition according to embodiments (1), (2), (3), (4), (5), (6), (9) or (10) in the preparation of a nutraceutical, dietary, pharmaceutical or cosmetic composition or product, particularly preparation of a medicament, in the treatment, lessening or prevention of a condition selected from the group consisting of migraine, preferably migraine headache, anxiety, irritable bowel syndrome, ocular hypotension, heart valve disease, and addiction/abuse of a member of the phenethylamine family of stimulants.
(12): A nutraceutical, dietary, pharmaceutical or cosmetic composition or product comprising a mixture or composition of embodiments (1), (2), (3), (4), (5) or (6).
(13): Composition or product according to embodiment (12) for treatment or prevention of migraine, anxiety, irritable bowel syndrome, ocular hypotension, heart valve disease, and addiction/abuse of a member of the phenethylamine family of stimulants.
(14): Composition or product according to embodiment (13) in the form suitable for oral administration.

The following non-limiting Examples are presented to better illustrate the invention.

### EXAMPLES

### Example 1: Antagonism of Human Serotonin 5-HT2B Using the IP1 Functional Assay

Retinoid mixture comprising retinyl acetate as defined herein can be produced as described in WO2022090549 (Ex.1), wherein the retinoids are accumulated in the lipid phase with subsequent isolation/extraction with ethanol or the like as known in the art.

The human 5-HT2B receptor expressed in transfected CHO cells is used to determine antagonistic effects by measuring the effects on agonist-induced inositol monophosphate (IP1) production using the HTRF® detection method (Porter et al., 1999, Brit. J. Pharmacol., 128: 13-20; Trinquet et al., 2006, Analytical Biochemistry 358, 126-135) The CHO cells can be suspended in a buffer containing 10 mM Hepes/NaOH (pH 7.4), 4.2 mM KCl, 146 mM NaCl, 1 mM CaCl₂, 0.5 mM MgCl₂, 5.5 mM glucose and 50 mM LiCl, then distributed in microtiter plates at a density of about 20000 cells/well and preincubated for 5 min at room temperature in the presence of buffer (basal control), test compound, i.e. reference samples and inventive mixture of retinyl acetate (“retAc”) (see Table 1) or a very potent known reference antagonist SB-206553. Thereafter, the reference agonist 5-HT (serotonin) is added at a final concentration of 30 nM. For basal control measurements, separate assay wells do not contain 5-HT. Following 30 min incubation at 37° C., the cells are lysed and the fluorescence acceptor (D2-labeled IP1) and fluorescence donor (anti-IP1 antibody labeled with europium cryptate) are added. After 60 min at room temperature, the fluorescence transfer is measured at wavelengths Ex=337 nm and Em=620 and 665 nm using a microplate reader (Envision, Perkin Elmer). The IP1 concentration can be determined by dividing the signal measured at 665 nm by that measured at 620 nm (ratio). The results are expressed as a percent inhibition of the control response to 30 nM 5-HT. Mean values, standard deviation are calculated and used in a dose response model to calculate the IC50 values.

TABLE 1
cis/trans ratio of tested retinyl acetate mixtures
(“RetAc mix”) and a control (“Reference
retAc”) as analyzed by HPLC. For further details see text.

| | Reference | |
|---|---|---|
| | retAc | RetAc mix |
| ratio cis/trans | 0.03 | 0.0025 |

The standard reference antagonist SB-206553 is tested in each experiment at several concentrations to generate a concentration-response curve. The dose response curve is used to calculate the IC50 value. The same procedure is done for RetAc mix and the Reference RetAc to determine the IC50 values. The results are shown in Table 2. When using the retinyl acetate mix according to the present invention, the IC50 values could be dramatically decreased, i.e. the antagonistic effect is dramatically increased as compared to the Reference retAc.

TABLE 2
IC50 values of RetAc mix (see Table 1) compared
to a reference sample (Reference retAc) or SB-206553
as indicated. For further details see text.

| Tested compound | IC50 | |
|---|---|---|
| SB-206553 (reference) | 26 | nM |
| Reference retAc | 12 | μM |
| RetAc mix | 5.2 | μM |

### Example 2: Formulations

Cosmetic formulations in the form of gels, emulsions, shampoo, hair cream, lotion, make-up, toner, after-shave, creams, hand sanitizer are created according to methods known in the art. If not indicated otherwise the pH of the formulations is adjusted to pH 3-7.5 as deemed appropriate. In the following formulations the term “NEW INGREDIENT” refers to retinyl acetate (RetAc mix) with a cis/trans ratio as indicated in Table 1.

TABLE 3
Retinyl acetate comprising gels. For further details see text.

| | 1 | 2 | 3 | 4 | 5 |
|---|---|---|---|---|---|
| Example: Gels | Wt.-% | | | | |
| Acrylates/Octylacrylamide | 1 | 1 | 1 | 1 | 1 |
| Copolymer | | | | | |
| Alcohol Denat. | 10 | 30 | 59 | 5 | 54 |
| Butylene Glycol Dicaprylate/ | | | 2 | 4 | |
| Dicaprate | | | | | |
| C₁₂₋₁₅ Alkyl Benzoate | 8.5 | 7.5 | | 2 | 5 |
| Phenylethylbenzoate | | | | 2.5 | |
| Cocoglyceride | | | 2 | 5 | |
| Tridecylsalicylate | 1.5 | 3 | 1 | | 3 |
| Hydroxypropylcellulose | 0.8 | 0.5 | 0.8 | 0.45 | 0.5 |
| Butyl Methoxydibenzoylmethane | | 2.5 | 4.5 | | 3 |
| 2.4.6-Tribiphenyl-4-yl-1.3.5 Triazine | | | 6 | | |
| Diethylamino Hydroxybenzoyl Hexyl | | 1.0 | | | |
| Benzoate | | | | | |
| Ethylhexyl Methoxycinnamate | | 6.5 | | | |
| Ethylhexyl Salicylate | | | | | 5 |
| Homosalate | | | 4.5 | | |
| Octocrylene | | 3.8 | | | 4 |
| Ethylhexyl Triazone | | 1 | | | |
| Drometrizole Trisiloxane | | 1 | | | 1 |
| NEW INGREDIENT | 0.5 | 0.3 | 0.1 | 0.05 | 0.15 |
| Bis-Ethylhexyloxyphenol | | | 1 | | |
| Methoxyphenyltriazine | | | | | |
| Vitamin E Acetate | | | 0.2 | 0.5 | |
| Niacinamide | | 2.0 | | | 3.0 |
| Glycerin | 8 | 5 | 4 | 6 | 2 |
| Ascorbylglucoside | 0.1 | | | 1.0 | |

| | |
|---|---|
| Fragrance. Colours | q.s. |
| Water | ad 100 |

TABLE 4
Retinyl acetate comprising O/W emulsions. Palmitoyl Oligopeptide & Palmitoyl
Tetrapeptide-7 are given in [ppm]. For further details see text.

| | 1 | 2 | 3 | 4 | 5 | 6 | 7 |
|---|---|---|---|---|---|---|---|
| Example: O/W Emulsions | Wt.-% | | | | | | |
| Glyceryl Stearate Citrate | 2 | 2 | 3 | | | | |
| Glyceryl Stearate SE | | | | 1 | 1 | 1.5 | |
| Cetearyl Alcohol + PEG-40 Rizinusoil + | | | | 2.5 | 2.5 | 3 | |
| Sodium Cetearyl Sulfate | | | | | | | |
| Potassium Cetyl Phosphate | | 0.5 | | | | | 2 |
| Cetearyl Alcohol | | | 1 | 1 | | | 2 |
| Stearyl Alcohol | 0.5 | | | | | 2 | |
| Coco caprylate/caprate | 1 | 1 | | | 3 | | |
| Acrylates/C₁₀₋₃₀ Alkyl Acrylate | 0.1 | 0.2 | | | 0.1 | | |
| Crosspolymer | | | | | | | |
| Carbomer | | 0.2 | 0.3 | 0.1 | | | |
| Xanthan Gum | 0.4 | | 0.2 | 0.2 | 0.3 | 0.4 | 0.2 |
| C₁₂₋₁₅ Alkyl Benzoate | | 3 | | | 5 | | 4 |
| 2-Phenylethylbenzoate | 5 | 2 | | | | | |
| Butylene Glycol Dicaprylate/Dicaprate | 5 | | | | 3 | 3 | |
| Tridecylsalicylate | | 2.5 | | 3 | | 15 | 5.9 |
| Dicaprylcaprate | 2 | 2 | | | 2 | 2 | |
| Cyclomethicone | | | | 5 | 10 | | 5 |
| Dimethicone | | | | | 5 | | |
| PVP Hexadecene Copolymer | | 0.5 | | | | 1 | 2 |
| Propylene Glycol | | | 1 | | 5 | 3 | |
| Ascorbylglucoside | | 0.1 | | | 0.5 | | |
| Niacinamide | 1 | | | | 2 | | |
| Tranexamic Acid | | | 3 | 0.5 | | 1.5 | |
| Glycerin | 3 | 3 | 7 | 5 | 13 | 3 | |
| Propanediol | | 2 | | 5 | | | 3 |
| Alcohol denat. | 2 | 3 | | 7 | | | |
| Titanium Dioxide | 3 | | | 2 | | | 3 |
| Phenylene-1.4-bis-(2-benzimidazyl)- | 3 | 2 | | | | | |
| 3.3-5.5-tetrasulfonic Acid | | | | | | | |
| Ethylhexyl Triazone | 2.5 | 2 | | 1 | | | 1 |
| Phenylbenzimidazole Sulfonic Acid | | | 2 | | | 1 | 2 |
| 4-Methoxycinnamate (2-ethylhexyl) ester | | 5 | | 2 | | | |
| Butyl Methoxydibenzoylmethane | 5 | 1 | | | 3 | | 4 |
| Ethylhexylsalicylate | 5 | | 0.5 | 4 | 5 | | 4 |
| Polysilicone-15 | | | 4 | | | | 1 |
| Isoamyl p-Methoxycinnamate | | 3 | 6 | | | | |
| Diethylamino Hydroxybenzoyl | | | | 6 | | | |
| Hexyl Benzoate | | | | | | | |
| Methylene Bis-Benztriazoyl | | | 5 | | | | |
| Tetramethylbutylphenol | | | | | | | |
| 1.1′-(1.4-piperazinediyl)bis[1- | | | 2 | | | | |
| [2-[4-(diethylamino)-2- | | | | | | | |
| hydroxybenzoyl]phenyl]-methanone | | | | | | | |
| Octocrylene | 3 | | | | 5 | 7 | 4 |
| Bis-Ethylhexyloxyphenol | | 1 | | 1 | | 1 | 0.5 |
| Methoxyphenyltriazine | | | | | | | |
| NEW INGREDIENT | 0.1 | 0.2 | 0.15 | 0.3 | 0.7 | 0.5 | 0.25 |
| Butyloctyl Methoxycrylene | | | | | 3 | | |
| Diethylhexyl Syringylidenemalonate | | | | | | | 2 |
| Vitamin E Acetate | 0.2 | 0.2 | 0.2 | 0.3 | 0.1 | 0.5 | 0.3 |
| Palmitoyl Oligopeptide & Palmitoyl | | 10 | | | 30 | | |
| Tetrapeptide-7 | | | | | | | |
| Disodium EDTA | 0.1 | 0.1 | 0.2 | 0.2 | 0.5 | 0.2 | 0.2 |
| DHA | | | | | 2 | | |
| Erythrulose | | | | 1.9 | | | |
| Silica | 1 | | 2.5 | | | 0.5 | |
| Methyl Methacrylate Crosspolymer | | | | 1 | | | 1 |
| Silica & Methicone | | 2 | | | 3 | | |

| | |
|---|---|
| Fragrance. Preservation agents | q.s |
| Colours. etc. | q.s |
| Citric Acid. Sodium Citrate | q.s |
| Sodium Hydroxide | q.s |
| Tromethamine | q.s |
| Water | ad 100 |

TABLE 5
Retinyl acetate comprising O/W emulsions. Palmitoyl Pentapeptide-4
is given in [ppm]. For further details see text.

| | 1 | 2 | 3 | 4 | 5 |
|---|---|---|---|---|---|
| Example: O/W Emulsions | Wt.-% | | | | |
| Glyceryl Stearate | 2.5 | 2 | 1.2 | 1 | |
| PEG-40 Stearate | 1 | | | | |
| PEG-100 Stearate | | 2.5 | | | |
| Ceteareth-20 | | | | | 1 |
| Potassium Cetyl Phosphate | | 0.5 | | | 1 |
| Stearic Acid | | | 2.5 | 3 | |
| Cetearyl Alcohol | 4 | | | 2 | |
| Stearyl Alcohol | | 2 | 1 | | |
| Cetyl Alcohol | | | 1 | 1 | |
| Acrylates/C₁₀₋₃₀ Alkyl | | | | 0.2 | 0.2 |
| Acrylate Crosspolymer | | | | | |
| Carbomer | 0.1 | | 0.2 | | |
| Xanthan Gum | | 0.3 | | | |
| C₁₂₋₁₅ Alkyl Benzoate | 5 | | | 2 | 5 |
| Petrolatum | 5 | | 3 | | |
| Butylene Glycol Dicaprylate/Dicaprate | | 4 | 2 | | 9 |
| Hydrogenated Polydecene | | | 3 | | 2 |
| Caprylic/Capric Triglyceride | 1 | 3 | | 5 | |
| Cyclomethicone | | 5 | 2 | | |
| Methylpropandiol | 2 | | | | 3 |
| Glycerin | 4 | 4 | 3 | | 3 |
| Propanediol | | 3 | | 3 | |
| Glyceryl Glucoside | | 0.5 | 1 | | |
| Alcohol denat. | | 3 | 0.5 | 10 | |
| Butylene Glycol | | | 3 | | |
| Ascorbylglucoside | | 0.5 | | 1.0 | |
| Ubiquinone (Coenzyme 10) | 0.1 | | 0.05 | | |
| Hyaluronic acid | | | | | 0.2 |
| Bisabolol | 0.5 | | | | |
| Palmitoyl Pentapeptide-4 | | | 20 | | |
| Isotridecylsalicylate | | | 1 | 3 | 5 |
| Titanium Dioxide | 1 | | 0.5 | 2 | |
| 2.4.6-Tribiphenyl-4-yl-1.3.5 Triazine | | | 0.5 | 4 | |
| Ethylhexyl Bis-Isopentylbenzoxazolylphe- | | 1 | | | 0.5 |
| nyl Melamine | | | | | |
| Ethylhexyl methoxycinnamate | | | | | 2 |
| Phenylbenzimidazole Sulfonic Acid | 1.5 | | | 2 | |
| Butyl Methoxydibenzoylmethane | 2.5 | 1 | | 2 | 2 |
| Methylbenzylidene Camphor | | | | | 2 |
| Disodium Phenyl Dibenzimidazole | | | 1 | | |
| Tetrasulfonic Acid | | | | | |
| NEW INGREDIENT | 0.1 | 0.15 | 0.25 | 0.3 | 0.5 |
| Octocrylene | | 5 | | | |
| Polysilicone-15 | | | | 2 | |
| Ethylhexyl Salicylate | 3 | | | | 5 |
| Homosalate | | | 4 | | 2 |
| Benzophenone-3 (Oxybenzone) | | 2 | | | |
| Drometrizole Trisiloxane | 0.5 | | | 1 | |
| Terephthalidene Dicamphor Sulfonic Acid | 1.5 | | | 0.5 | |
| Butyloctyl Methoxycrylene | | 1 | | | |
| Silica | 1 | | 2.5 | | |
| Silica & Methicone | | 4 | | 1 | 2.5 |
| Methyl Methacrylate Crosspolymer | | | | 1 | |

| | |
|---|---|
| Disodium EDTA | 0.1 |
| Fragrance. Preservatives | q.s |
| Sodium Hydroxide | q.s |
| Water | Ad 100 |

TABLE 6
Retinyl acetate comprising W/O emulsions. Palmitoyl Tripeptide-38
is given in [ppm]. For further details see text.

| | 1 | 2 | 3 | 4 | 5 |
|---|---|---|---|---|---|
| Example: W/O Emulsions | Wt.-% | | | | |
| Polyglyceryl-2 Dipoly-hydroxystearate | 3 | 5 | 3 | | |
| PEG-30 Dipoly-hydroxystearate | | | 2 | 3 | 4 |
| Sodium Starch Octenylsuccinate | 0.5 | 0.4 | 0.6 | 0.3 | 0.5 |
| Glycine | | | 0.5 | 0.4 | |
| Alcohol denat. | | 5 | 2 | | 8 |
| Magnesium Sulfate | 0.2 | 0.3 | 0.3 | 0.4 | 0.5 |
| C₁₂₋₁₅ Alkyl Benzoate | 5 | 3 | | | 5 |
| Triheptanoin | | 2 | | | |
| Butyleneglycol Dicaprylat/Dicaprate | 5 | | | | 3 |
| Dicaprylyl Ether | | | | | 2 |
| Mineral Oil | | 4 | | 6 | |
| Octyldodecanol | 2 | | | | |
| Dicapryl Caprate | | 2 | | | 2 |
| Cyclomethicone | 5 | | 5 | 10 | |
| Dimethicone | | | | 5 | |
| Isohexadecane | | 1 | | | |
| Butylene Glycol | 5 | 8 | | | |
| Glycerin | | 5 | 7 | 5 | 3 |
| Propanediol | 3 | | | 5 | |
| Tridecylsalicylate | | 1 | 2 | | |
| 2-Phenylethyl-benzoate | | | 2 | | 4 |
| Isopropyl Lauroyl Sarcosinate | | | 1 | | 2 |
| Ethylhexylmethoxycinnamate | 2 | | | | |
| Ethylhexyl Triazone | 2 | 3 | 3 | | |
| Ethylhexyl Bis-Isopentyl- | | | | 3 | |
| benzoxazolylphenyl Melamine | | | | | |
| Diethylhexyl Butamido Triazone | | 1.5 | 1.5 | | |
| Butyl Methoxy dibenzoylmethane | 1 | 4 | 3 | | 5 |
| Methylbenzylidene Camphor | | | | | 1 |
| 2.4.6-Tribiphenyl-4-yl-1.3.5 Triazine | | | 5 | | |
| 2-(4-Diethylamino-2-hydroxybenzoyl)- | | | | 2 | |
| Benzoic Acid Hexylester | | | | | |
| NEW INGREDIENT | 0.2 | 0.5 | 0.1 | 0.2 | 0.3 |
| Palmitoyl Tripeptide-38 | | 5 | | | |
| Titanium Dioxide (Parsol TX) | 5 | | 2 | | 3 |
| Polysilicone-15 | 2 | | | | 1.5 |
| Octocrylene | | 3.6 | | | |
| Ethylhexyl Salicylate | | | | | 5 |
| Phenylbenzimidazole Sulfonic Acid | 3 | | | | |
| Bis-Ethylhexyl-oxyphenol Methoxy- | 1 | 2 | 2 | | 2 |
| phenyltriazine | | | | | |
| Methylene Bis-benzotriazolyl | 2 | | 3 | | 1 |
| tetramethylbutylphenol | | | | | |
| 1.1′-(1.4-piper-azinediyl)bis[1-[2-[4- | | | | 2 | |
| (diethylamino)-2-hydroxybenzoyl] phenyl]- | | | | | |
| methanone | | | | | |
| Butyloctyl Methoxycrylene | | 4 | | | |
| Vitamin E Acetate | 0.2 | 0.2 | 0.2 | 0.3 | 0.1 |
| Ascorbylglucoside | 0.2 | | 0.5 | | |
| Disodium EDTA | 0.1 | 0.1 | 0.2 | 0.2 | 0.2 |

| | |
|---|---|
| Fragrance. Preservatives | q.s |
| Water | ad 100 |

TABLE 7
Retinyl acetate comprising O/W emulsion for
facial cream. For further details see text.

| | |
|---|---|
| 1 | 2 |

| | |
|---|---|
| Example: O/W-Emulsion: facial cream | Wt.-% |

| | | |
|---|---|---|
| Polyglyceryl-3-Methylglucosedistearate | 2.0 | 2.0 |
| Sorbitanstearate | 3.0 | 3.0 |
| Cetylalcohol | 2.0 | 2.0 |
| Myristylmyristate | 1.0 | 1.0 |
| Dicaprylylether | 3.0 | 3.0 |
| Octyldodecanol | 2.0 | 2.0 |
| C₁₂₋₁₅ Alkylbenzoate | 3.0 | 3.0 |
| Cetearyl Ethylhexanoat + Isopropylmyristate | 2.0 | 2.0 |
| Octocrylene | 2.0 | 2.0 |
| Ethylhexyltriazone | 1.0 | 1.0 |
| Butyl Methoxydibenzoxylmethane | 2.0 | 2.0 |
| Magnesium Aluminium Silicate | 0.2 | 0.2 |
| Glyceryl Glucoside | 2.0 | 3.0 |
| Glycerine | 5.0 | 5.0 |
| Phenoxyethanol | 0.4 | 0.4 |
| Parabene | 0.3 | 0.3 |
| Vitamin E Acetate | 0.1 | 0.1 |
| Hyaluronic Acid | 0.5 | 0.2 |
| NEW INGREDIENT | 0.6 | 0.1 |
| BHT | 0.05 | 0.05 |
| EDTA | 0.2 | 0.2 |
| Carbomer | 2.0 | 0.2 |
| Perfume | q.s. | q.s. |
| Water | ad 100 | ad 100 |

TABLE 8
Retinyl acetate comprising shampoo. For further details see text.

| Example: Shampoo | % w/w |
|---|---|
| Sodium Laureth Sulfate | 10.0 |
| Ammonium Lauryl Sulfate | 5.0 |
| Water | 40.0 |
| Cocamidopropyl Betaine | 2.5 |
| Borago Officinalis Seed Oil & Tocopherol & Ascorbyl Palmitate | 0.3 |
| PEG-40 Hydrogenated Castor Oil | 1.0 |
| Fragrance | 0.3 |
| Panthenol | 1.0 |
| Disodium EDTA | 0.1 |
| Water | 10.0 |
| Sodium benzoate | 0.5 |
| Citric acid | 0.2 |
| NEW INGREDIENT | 0.05 |
| Sodium Chloride | 2.0 |
| PEG-150 Pentaerythrityl Tetrastearate | 3.0 |
| Water | ad. 100 |

TABLE 9
Retinyl acetate comprising anti dandruff
shampoo. For further details see text.

| Example: Anti Dandruff Shampoo | % w/w |
|---|---|
| Aqua (water) | Ad 100 |
| Ammonium laureth sulfate | 10.0 |
| Ammonium lauryl sulfate | 5.0 |
| Glycol distearate | 1.0 |
| Dimethicone | 1.0 |
| Cetyl alcohol | 0.5 |
| Cocamide MEA | 3.0 |
| Zinc Pyrithione | 1.0 |
| Zinc Sulfate | 0.2 |
| TEA-dodecylbenzenesulfonate | 0.2 |
| TEA-Sulfate | 0.15 |
| Guar hydroxypropyltrimonium chloride | 0.2 |
| Hydrogenated polydecene | 1.0 |
| Polyquaternium 10 | 0.3 |
| PEG-7M | 0.5 |
| PEG-40 Hdrogenated Castor Oil | 1.0 |
| Trimethylpropane tricaprylate/tricaprate | 1.0 |
| Pyridoxine HCL | 0.1 |
| Preservative | q.s. |
| Fragrance | 0.3 |
| E 104. E 110. E 132 | 0.02 |
| NEW INGREDIENT | 0.1 |

TABLE 10
Retinyl acetate comprising revitalizing
hair cream. For further details see text.

| Example: Revitalizing Hair Cream | % w/w |
|---|---|
| Simmondsia Chinensis (Jojoba) Seed Oil | 3.0 |
| Prunus Armeniaca (Apricot) Kernel Oil | 3.0 |
| Phenyl Trimethicone | 2.0 |
| C12-15 Alkyl Benzoate | 2.0 |
| Glyceryl Stearate SE | 2.0 |
| Polysilicone-15 | 0.5 |
| Tocopheryl Acetate | 0.5 |
| Cetearyl Alcohol | 1.6 |
| Polyquaternium-7 | 1.0 |
| NEW INGREDIENT | 0.1 |
| Aqua | Ad. 100 |
| Behentrimonium Chloride | 1.0 |
| Cocodimonium Hydroxypropyl Hydrolyzed Wheat Protein | 0.3 |
| Propylene Glycol (and) Diazolidinyl Urea (and) Methylparaben | 1.0 |
| (and) Propylparaben | |

TABLE 11
Retinyl acetate comprising natural lotion.
For further details see text.

| Example: Natural Lotion | % w/w |
|---|---|
| ISOPROPYL PALMITATE | 3 |
| PRUNUS AMYGDALUS DULCIS OIL | 1.5 |
| CAPRYLIC/CAPRIC TRIGLYCERIDE | 6 |
| CETEARYL ALCOHOL | 0.6 |
| Helianthus Annuus (Sunflower) Seed Oil | 2 |
| Sodium Benzoate | 0.25 |
| Potassium Sorbate | 0.15 |
| AQUA | Ad 100 |
| CETEARYL GLUCOSIDE | 3 |
| GLYCERIN | 3 |
| Rosa Gallica Flower Extract | 5.0 |
| bentonite | 1 |
| XANTHAN GUM | 0.2 |
| AQUA | 30 |
| LEONTOPODIUM ALPINUM EXTRACT | 2.5 |
| Sodium Hydroxide | 0.3 |
| NEW INGREDIENT | 0.2 |
| TOCOPHEROL | 0.1 |

TABLE 12
Retinyl acetate comprising liquid make-up. For further details see text.

| | 1 | 2 | 3 | 4 | 5 | 6 | 7 | 8 |
|---|---|---|---|---|---|---|---|---|
| Example: Liquid make-up | Wt.-% | | | | | | | |
| Water (Distilled) | Ad 100 | | | | | | | |
| Xanthan Gum | 0.3 | 0.33 | 0.33 | 0.1 | 0.33 | 0.33 | 0.33 | 0.33 |
| Cellulose Gum | 0.2 | 0.23 | 0.23 | 0.5 | 0.23 | 0.5 | 0.23 | 0.23 |
| NEW INGREDIENT | 0.3 | 0.1 | 0.1 | 0.15 | 0.25 | 0.01 | 0.1 | 0.05 |
| Triethanolamine | 0.7 | | | | | | | |
| PEG 1450 | 2.35 | | | | | | | |
| Methyl Paraben | 0.28 | | | | | | | |
| Pentylene Glycol | 1.0 | 2.0 | 1.7 | 1.88 | | | | |
| Polysorbate 60 | 0.47 | | | | | | | |
| Titanium Dioxide | 8.44 | 7.0 | 9.0 | 8.5 | 8.44 | | | |
| Brown Iron Oxide | 2.35 | | | | | | | |
| Yellow BC Iron Oxides | 0.94 | | | | | | | |
| Orange Wax | 6.1 | | | | | | | |
| Cera Bellina | 4.69 | | | | | | | |
| Octyl Palmitate | 6.57 | | | | | | | |
| Silicone 245 | 2.0 | 2.0 | | | | | | |
| Cyclopentasiloxane | 1.0 | 1.0 | | | | | | |
| Dimethiconol | 0.20 | | | | | | | |
| Isopropyl Palmitate | 6.1 | 6.1 | 6.1 | | | 1.0 | | |
| C13-15 Alkane | | | 2.0 | 1.55 | 2.8 | | 2.0 | |
| C15-20 Alkane | | | 1.0 | 2.0 | 2.0 | | | |
| C15-19 Alkane | | 6.0 | | | 3.0 | | | |
| Cocunut Alkanes | | | | | | 5.1 | 2.1 | |
| Hydrogenated Castor Oil | 1.41 | | | | | | | |
| Isostearic Acid | 1.5 | | | | | | | |
| Jojoba wax | 0.94 | | | | | | | |
| Dextrin Palmitate | | 4.0 | | | 2.0 | | | 2.0 |

| | |
|---|---|
| Propylene Glycol Stearate | 1.89 |
| Propyl Paraben | 0.28 |

TABLE 13
Retinyl acetate comprising moisturizing toner. For further details see text.

| | 1 | 2 | 3 | 4 | 5 | 6 | 7 | 8 |
|---|---|---|---|---|---|---|---|---|
| Example: Moisturizing toner | Wt.-% | | | | | | | |
| Butylene Glycol | 5 | 4 | 3 | 2 | 1 | 2 | | |
| Phenoxyethanol; Ethylhexyl glycerin | | | | | | | 0.8 | 0.5 |
| Caprylyl-Glycol | 0.5 | 0.1 | 0.25 | 0.1 | 0.2 | | | |
| Hexylene Glycol | 1 | 2 | 1.5 | | 1 | 1 | | |
| Propylene glycol | 1 | 2 | 3 | 4 | 5 | | | |
| Dipropylene glycol | | | | | | 1 | 5 | 3 |
| Sorbitol | 1 | 3 | 0.5 | | | | 1 | 1 |
| Panthenol | 0.1 | 1 | 1.5 | | | | | |
| Lactic acid | 1 | 0.5 | | | | | | |
| Sodium Gluconate | 0.05 | 0.1 | 0.2 | | | | | |
| Betaine | 0.5 | 1 | | | | | 0.2 | |
| Sodium Phytate | | | | 0.05 | 0.1 | 0.2 | | |
| Trehalose | | | | 1 | 2 | 0.5 | | |
| Mannitol | | | | | | | 1 | 2 |
| Capryloyl Glycine | | | | | 0.1 | 0.5 | 1.0 | |
| Honey/Mel | | | | 0.1 | 1 | | 2 | |
| alcohol | | | 2 | 1 | | | | |
| Benzyl Alcohol | | | | | | | 0.3 | 1 |
| Hydroxyacetophenone | | | | | 0.5 | 1 | 0.1 | 0.3 |
| Bis-PEG-18 Methyl Ether Dimethyl | 0.1 | 0.2 | 0.5 | 0.15 | | | | |
| Silane | | | | | | | | |
| Sodium Hyaluronate | 0.01 | 0.2 | | | | | | |
| Biosaccharide Gum 1 | | | 0.1 | 1 | 2 | | | |
| Saccharide Isomerate | 0.5 | | 3 | | 1 | | | |
| Yeast extract | | | 0.01 | 0.2 | | | | |
| Algae extract | 0.01 | | | | | 0.1 | | 0.2 |
| Lactobacillus Ferment Lysate | | 0.3 | | 0.1 | | 0.01 | | |
| NEW INGREDIENT | 0.1 | 0.01 | 0.3 | 0.5 | 0.6 | 0.08 | 0.2 | 0.25 |
| Vitamin E Acetate | 0.1 | | | 0.5 | | 0.2 | 0.5 | |
| Glycerin | 1 | 6 | 2 | 5 | 3 | 4 | 1 | 2.5 |
| Polysorbate-20 | 0.5 | | | | | | | |
| PEG-40 Hydrogenated Castor oil | | | | 0.2 | 0.5 | 0.1 | | |
| PEG-60 Hydrogenated Castor oil | 0.2 | 0.4 | | | | | | |
| PEG-8 | | | | | | | 0.2 | 0.5 |
| Polyglyceryl-6 Caprylate (and) | | | | | | | 0.2 | 0.5 |
| Polyglyceryl-3 Cocoate (and) | | | | | | | | |
| Polyglyceryl-4 Caprate (and) | | | | | | | | |
| Polyglyceryl-6 Ricinoleate | | | | | | | | |
| Polyglyceryl-3 | | 0.2 | 0.5 | | | | | |
| Caprate/Caprylate/Succinate | | | | | | | | |

| | |
|---|---|
| Fragrance. Colours | q.s |
| Water | ad 100 |

TABLE 14
Retinyl acetate comprising after shave
SPF 15. For further details see text.

| Example: After Shave SPF 15 | % w/w |
|---|---|
| Aqua | Ad 100 |
| Propylene Glycol | 5.0 |
| Butylene Glycol | 5.0 |
| Acrylates/C10-30 Alkyl Acrylate Crosspolymer | 0.3 |
| Disodium Edta | 0.1 |
| Butyl Methoxydibenzoylmethane | 4.0 |
| Octocrylene | 4.0 |
| Polysilicone-15 | 2.0 |
| Ethylhexyl Salicylate | 5.0 |
| Homosalate | 8.0 |
| Neopentyl Glycol Diheptanoate. Propylene Glycol Dibenzoate | 3.0 |
| Phenoxyethyl Caprylate | 2.0 |
| Dextrin Palmitate, C15-19 Alkane | 3.0 |
| Silica | 2.0 |
| Alcohol Denat . . . Aqua | 10.0 |
| Phenoxyethanol. Caprylyl Glycol | 0.5 |
| NEW INGREDIENT | 0.1 |
| Perfume | 0.15 |
| Sodium Hydroxide | qs. |

TABLE 15
Retinyl acetate comprising anti-aging serum. For further details see text.

| Example: Anti-aging Serum | % w/w | | | | | |
|---|---|---|---|---|---|---|
| Acrylates/C10-30 Alkyl | 0.3 | | | | | |
| Acrylate Crosspolymer | | | | | | |
| Glyceryl Stearate Citrate | 2.5 | | | | | |
| Coco-Caprylate | 3.0 | | | | | |
| Hydrogenated Coco-Glycerides | 2.5 | | | | | |
| Neopentyl Glycol Diheptanoate | 2.0 | | | | | |
| Dicaprylyl Ether | 2.0 | | | | | |
| Preservative | q.s. | | | | | |
| Glycerin | 2.0 | | | | | |
| Aqua | 82.2 | | | | | |
| Sodium Hydroxide | 0.5 | | | | | |
| NEW INGREDIENT | 0.05 | 0.10 | 0.15 | 0.20 | 0.25 | 0.30 |

TABLE 16
Retinyl acetate comprising anti-aging cream. For further details see text.

| Example: Anti-aging Cream | % w/w | | | | | | |
|---|---|---|---|---|---|---|---|
| Potassium Cetyl Phosphate | 1.5 | | | | | | |
| Cetyl Alcohol | 2.0 | | | | | | |
| Stearyl Alcohol | 3.0 | | | | | | |
| Hydrogenated Coco-Glycerides | 4.0 | | | | | | |
| Butyrospermum Parkii (Shea Butter) | 1.5 | | | | | | |
| Lanolin Alcohol | 1.0 | | | | | | |
| Cetearyl Isononanoate | 1.0 | | | | | | |
| Caprylic/Capric Triglyceride | 2.0 | | | | | | |
| Ethylhexyl Palmitate | 6.0 | | | | | | |
| C12-15 Alkyl Benzoate | 5.0 | | | | | | |
| Macadamia Ternifolia Seed Oil | 1.0 | | | | | | |
| Carbomer | 0.3 | | | | | | |
| Glycerin | 6.0 | | | | | | |
| Butylene Glycol | 3.0 | | | | | | |
| Aqua | ad 100 | | | | | | |
| Palmitoyl Dipeptide-5 | 2.0 | | | | | | |
| Diaminobutyroyl Hydroxythreonine | | | | | | | |
| & Palmitoyl Dipeptide-5 | | | | | | | |
| Diaminohydroxybutyrate & Glycerin | | | | | | | |
| & Aqua | | | | | | | |
| NEW INGREDIENT | 0.05 | 0.10 | 0.12 | 0.15 | 0.20 | 0.25 | 0.30 |

| | |
|---|---|
| Preservative | q.s. |

TABLE 17
Retinyl acetate comprising hand sanitizer
gel. For further details see text.

| Example: Hand sanitizer gel | % w/w |
|---|---|
| Aqua | Add 100 |

| | | | | |
|---|---|---|---|---|
| Carbomer | 0.8 | 0.7 | 0.4 | 0.7 |
| Ethanol | 70.0 | 60.0 | 65.0 | |
| Isopropanol | | 5.0 | | 70.0 |
| NEW INGREDIENT | 0.05 | 0.10 | 0.20 | 0.03 |
| Tetrahydroxylpropyl | 0.3 | 0.3 | 0.2 | 0.45 |
| Ethylenediamine | | | | |

1. A retinoid mixture comprising cis and trans isomers of retinyl ester, particularly cis and trans isomers of retinyl acetate, wherein the cis/trans ratio within said mixture is 1 to more than 33.33, wherein the ratio is calculated as wt % of cis-isomers compared to wt % of trans-isomers based on total retinoids in said mixture, particularly wherein the mixture comprises less than 0.5 wt % of cis retinyl esters, preferably cis-retinyl acetate, based on total retinoids.

2. The retinoid mixture according to claim 1, comprising cis- and trans isomers of retinyl acetate, with a ratio of 1:40, 1:45, 1:50, 1:55, 1:60, 1:65, 1:66.67, 1:70, 1:75, 1:80, 1:90, 1:95, 1:100, 1:101.01, 1:105.26, 1:110, 1:111.11, 1:115, 1:117.65, 1:120, 1:125, 1:130, 1:133.33, 1:135, 1:140, 1:142.86, 1:145, 1:150, 1:155, 1:160, 1:165, 1:166.67, 1:170, 1:175, 1:180, 1:181.82, 1:185, 1:190, 1:195, 1:200, 1:204.08, 1:205, 1:210, 1:215, 1:220, 1:222.22, 1:225, 1:230, 1:232.56, 1:235, 1:240, 1:245, 1:250, 1:255, 1:260, 1:265, 1:270, 1:275, 1:280, 1:285, 1:285.71, 1:290, 1:295, 1:300, 1:305, 1:310, 1:315, 1:320, 1:325, 1:330, 1:333.33, 1:335, 1:340, 1:345, 1:350, 1:355, 1:357.14, 1:360, 1:365, 1:370, 1:375, 1:380, 1:385, 1:390, 1:395 or 1:400, 1:405, 1:410, 1:420, 1:430, 1:440, 1:450 or 1:500, 1:1000 or less, more preferably ranges of 1:33 to 1:1000, 1:100 to 1:500, 1:100 to 1:1000, 1:125 to 1:1000, 1:200 to 1:1000, 1:200 to 1:500, 1:200 to 1:400.

3. The retinoid mixture according to claim 2, wherein the retinoid mixture comprising cis- and trans retinyl acetate with a ratio of about 1:400.

4. The mixture according to claim 1 comprising 2 wt % or less retinol based on total retinoids.

5. The mixture according to claim 1 comprising 1 wt % or less retinal based on total retinoids.

6. The mixture according to claim 1 with a color value of L* being in the range of 50 to 100, a* being in the range of −25 to 10 and b* being in the range of 40 to 150.

7. A pharmaceutical, food, feed or cosmetic composition comprising a biologically-derived mixture of claim 1.

8. The composition according to claim 7, wherein the biologically-derived mixture is biologically produced through fermentation process.

9. The composition according to claim 7, wherein the mixture has an anthropogenic CO₂ emission profile of zero upon biodegradation.

10. The composition according to claim 7 for use as serotonin 5-HT2B receptor antagonist.

11. The composition according to claim 7 for use in preparation of a nutraceutical, dietary, pharmaceutical or cosmetic product, particularly preparation of a medicament, in the treatment, lessening or prevention of a condition selected from the group consisting of migraine, preferably migraine headache, anxiety, irritable bowel syndrome, ocular hypotension, heart valve disease, and addiction/abuse of a member of the phenethylamine family of stimulants.

12. A nutraceutical, dietary, pharmaceutical or cosmetic product comprising a mixture or composition according to claim 1.

13. Composition or product according to claim 7 for treatment or prevention of migraine, anxiety, irritable bowel syndrome, ocular hypotension, heart valve disease, and addiction/abuse of a member of the phenethylamine family of stimulants.

14. Method for prevention, treatment or lessening of symptoms associated with migraine, anxiety, irritable bowel syndrome, ocular hypotension, heart valve disease, and addiction/abuse of a member of the phenethylamine family of stimulants comprising administering a composition according to claim 7.
